(19) United States
(12) Patent Application Publication
(10) Pub. No.: US 2023/0134704 A1
(43) Pub. Date: May 4, 2023

(54) A METHOD OF TREATING CANCER BY UPREGULATING CATHELICIDIN GENE EXPRESSION AND INFUSING NATURAL KILLER CELLS

(71) Applicant: Annelise E. BARRON, Redwood City, CA (US)

(72) Inventors: Annelise E. Barron, Redwood City, CA (US); John A. FORTKORT, Austin, TX (US)

(21) Appl. No.: 17/800,903

(22) PCT Filed: Mar. 19, 2021
(86) PCT No.: PCT/US21/18902

§ 371 Date: Aug. 19, 2022

Related U.S. Application Data
(60) Provisional application No. 62/978,754, filed on Feb. 19, 2020.

(51) Int. Cl. A61K 35/17; C12N 5/0783; A61K 31/19; A61K 31/192; A61K 31/12; A61K 31/05; A61K 31/07; A61K 31/015; A61K 31/045; A61K 31/44; A61K 31/202; A61K 31/20

(52) U.S. Cl. CPC A61K 35/17; C12N 5/0646; A61K 31/19; A61K 31/192; A61K 31/12; A61K 31/05; A61K 31/07; A61K 31/015; A61K 31/045; A61K 31/44; A61K 31/202; A61K 31/20

(57) ABSTRACT

A method for treating a subject suffering from cancer is provided. The method includes diagnosing the subject as suffering from cancer; obtaining Natural Killer (NK) white blood cells; upregulating the cathelicidin gene CAMP in the subject; and infusing the NK cells into the body of the subject.

### CROSS-REFERENCE TO RELATED APPLICATION

The present application is a national stage filing of PCT/US21/18902, filed on Feb. 19, 2021, which has the same title and the same inventors, and which is incorporated herein by reference in its entirety; which claims the benefit of priority of U.S. patent application No. 62/978,754, filed Feb. 19, 2020, having the same inventors and entitled “A METHOD OF TREATING CANCER BY UPREGULATING CATHELICIDIN GENE EXPRESSION AND INFUSING NATURAL KILLER CELLS,” which is incorporated herein by reference in its entirety.

### FIELD OF THE DISCLOSURE

The present disclosure relates generally to methods for treating cancer, and more particularly to methods for treating pediatric cancers such as pediatric diffuse gliomas, by upregulating cathelicidin in a patient, either alone or as an adjuvant to administering NK cell therapy to the patient.

### BACKGROUND OF THE DISCLOSURE

Brain tumors are the most common solid tumors encountered in pediatric cancer patients, and the main cause of cancer-related death in children. Gliomas (brain tumors derived from glial cells) represent approximately 60% of all pediatric brain tumors, about half of which are characterized as high-grade gliomas (HGGs). See Rizzo D, Ruggiero A, Martini M, Rizzo V, Maurizi P, Riccardi R., “Molecular Biology in Pediatric High-Grade Glioma: Impact on Prognosis and Treatment”, Biomed Res Int. 2015; 2015:215135. Such pediatric high-grade gliomas (pHGGs), which include histone H3 K27M (H3K27M) mutated diffuse midline gliomas (DMGs), are aggressive, solid tumors that, together, account for approximately 8-12% of all central nervous system (CNS) tumors that arise in children. See Martinez-Vélez, N., Garcia-Moure, M., Marigil, M. et al., “The oncolytic virus Delta-24-RGD elicits an antitumor effect in pediatric glioma and DIPG mouse models”. Nat. Commun. 10, 2235 (2019). The standard treatment for many pHGGs is maximal surgical resection, followed by radiotherapy and/or temozolomide cycles. See Stupp, R. et al., “Effects of radiotherapy with concomitant and adjuvant temozolomide versus radiotherapy alone on survival in glioblastoma in a randomized phase III study: 5-year analysis of the EORTC-NCIC trial”. Lancet Oncol. 10, 459-466 (2009).

Due to their diffuse nature, surgery is not a suitable treatment option for DMGs. Consequently, radiotherapy (RT) remains the current de facto standard for treating these gliomas. Although RT may improve the quality of life and survival of DIPG patients, it is not a cure. Consequently, despite the fact that a significant number of clinical trials have been conducted which have investigated RT treatment of pHGG in general and DMGs in particular, the prognosis for patients suffering from these diseases has continued to be bleak. See Kline, C., Felton, E., Allen, I. E., Tahir, P. & Mueller, S. “Survival outcomes in pediatric recurrent high-grade glioma: results of a 20-year systematic review and meta-analysis”. J. Neurooncol. 137, 103-110 (2017). See also Hassan, H., Pinches, A., Picton, S. V. & Phillips, R. S. “Survival rates and prognostic predictors of high grade brain stem gliomas in childhood: a systematic review and meta-analysis”. J. Neurooncol. 135, 13-20 (2017). In particular, children suffering from pHGG have a 5-year survival of less than 20%. The prognosis for children diagnosed with DMGs is even worse: only 10% of these patients survive for 2 years following their diagnosis, and less than 1% survive for 5 years (see defeatdipg.org). This tragic state of affairs is evidenced by the proliferation of private foundations seeking to fund research for new treatments or cures for these cancers. Many of these foundations have been started by family members of those whose lives have been lost to these diseases. See, for example, the Gunnar Heinrich Foundation (www.gunnarfoundation.org).

Various cancer treatments have been developed in the art, some of which have been applied to diffuse midline gliomas. These include, for example, Chimeric Antigen Receptor, or CAR T-cell therapy. In particular, many patient-derived H3K27M-mutant glioma cell cultures have been found to exhibit uniform, high expression of the disialoganglioside GD2. Bioengineered and expanded anti-GD2 CAR T-cells incorporating a 4-1BBz costimulatory domain have been found to demonstrate antigen-dependent cytokine generation and DMG cell killing in vitro. See Mount C W, Majzner R G, Sundaresh S, et al. “Potent antitumor efficacy of anti-GD2 CAR T cells in H3-K27M+ diffuse midline gliomas”. Unfortunately, although systemic administration of GD2-CAR T-cells has been observed to clear engrafted tumors in some animal models, it is an inherently expensive treatment option. Moreover, as with other applications of CAR T-cell therapy, neuroinflammation is a common side effect of this treatment, which can result in lethal hydrocephalus in a significant percentage of the subjects to whom the treatment is applied. In addition, relapse is common in subjects treated with CAR T-cell therapy, possibly because the treatment does not remove cancer cells that express low amounts of the target surface marker. For example, treatment of subjects with GD2-CAR T-cells is known to leave behind a small number of residual GD2-low glioma cells, which can then serve as nucleating sites for regrown diffuse tumors.

There is thus a need in the art for new methods of treating pediatric brain tumors in general, and gliomas (including pHGG and DMGs) in particular. There is further a need in the art for such methods which are cost effective and have reduced negative side effects compared to alternative treatments in the art. These and other needs may be addressed with the systems and methodologies disclosed herein.

### SUMMARY OF THE DISCLOSURE

In one aspect, a method is provided for treating a subject suffering from cancer. The method comprises (a) diagnosing the subject as suffering from cancer; (b) upregulating the cathelicidin gene CAMP in the subject; (c) obtaining Natural Killer (NK) white blood cells; and (d) infusing the NK cells into the body of the subject.

In another aspect, a method for treating a subject suffering from cancer is provided. The method comprises (a) diagnosing the subject as suffering from cancer; (b) treating the cancer; and (c) upregulating the cathelicidin gene CAMP in the subject.

In a further aspect, a method for treating a subject suffering from cancer is provided. The method comprises (a) diagnosing the subject as suffering from a cancer characterized by at least one solid tumor; (b) treating the cancer; and (c) upregulating the cathelicidin gene CAMP in the subject.

In still another aspect, a method is provided for treating a subject suffering from cancer. The method comprises (a) diagnosing the subject as suffering from brain cancer; (b) causing the subject to undergo (a) an exercise therapy; and (c) administering a pharmaceutical composition to the subject, wherein the pharmaceutical composition includes at least two substances selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, genistein and pharmaceutically acceptable salts thereof.

In yet another aspect, a method is provided for treating a subject suffering from cancer. The method comprises (a) diagnosing the subject as suffering from brain cancer; (b) causing the subject to undergo a therapy selected from the group consisting of (i) sauna therapy and (ii) hydrotherapy, wherein said therapy includes exposing the subject to a temperature of at least 74° C.; and (c) administering a pharmaceutical composition to the subject, wherein the pharmaceutical composition includes at least two substances selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, genistein and pharmaceutically acceptable salts thereof.

### BRIEF DESCRIPTION OF THE DRAWINGS

FIG. 1 is a flowchart illustrating some embodiments of a method for NK cell therapy in accordance with the teachings herein.

FIG. 2 is an illustration of various ways in which NK cells may be utilized in immunotherapy.

### DETAILED DESCRIPTION

It has now been found that some or all of the foregoing needs may be met with the systems and methodologies disclosed herein. In a preferred embodiment, a method is provided for treating cancer patients in general, and patients suffering from brain cancers, including pediatric gliomas (including pHGG and DIPG), in particular. The method includes upregulating the cathelicidin gene CAMP in the subject as an adjuvant to Natural Killer (NK) cell therapy as, for example, by performing the upregulation before, during or after infusion of NK white blood cells into the body of the subject. The NK cells may then remediate the cancer through their native tumoricidal properties.

Without wishing to be bound by theory, the upregulation of the cathelicidin gene CAMP in the subject as an adjuvant to NK cell therapy is believed to significantly enhance the efficacy of the NK cell therapy. This approach is inherently less expensive than other treatment options such as CAR T-cell therapy, is conducive to outpatient administration, and is not characterized by common CAR T-cell side effects such as neuroinflammation and a significant mortality rate. Moreover, the inherent safety of this approach allows the upregulation of the cathelicidin gene CAMP in the subject to be continued indefinitely after NK T-cell therapy which, in addition to the greater efficacy of the NK T-cell therapy as promoted by upregulation of the cathelicidin gene CAMP in the subject before or during NK cell therapy, may help to prevent relapse.

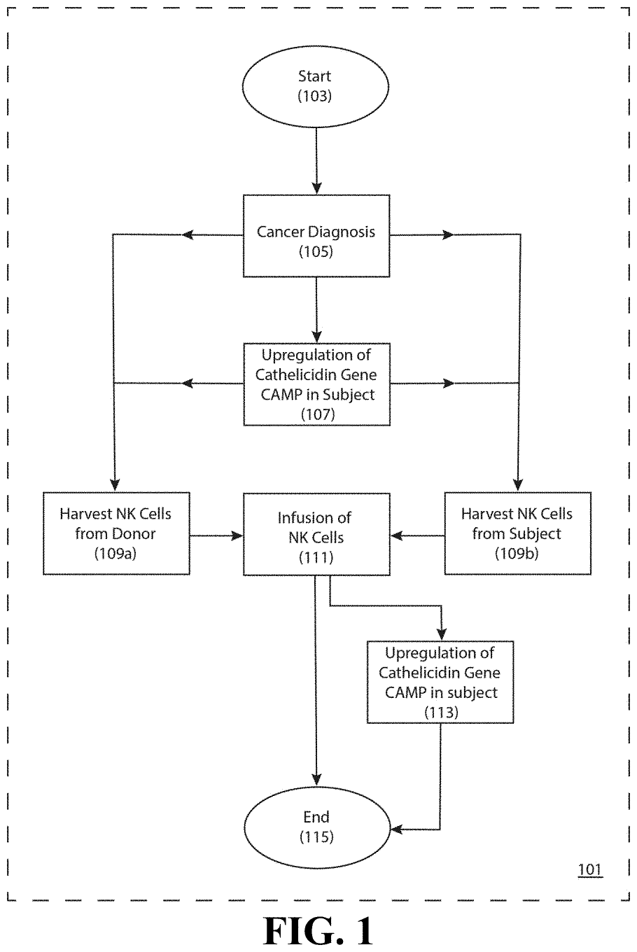

FIG. 1 depicts a first particular, non-limiting embodiment of a method in accordance with the teachings herein for treating a cancer patient. As seen therein, the method 101 commences 103 with a cancer diagnosis 105. The cancer diagnosis will typically be made by an oncologist or other suitable healthcare provider and may include, for example, a conclusion that the subject is suffering from cancer (such as, for example, a pediatric glioma such as pHGG or DIPG). A first suitable upregulating means is then employed to upregulate the cathelicidin gene CAMP in the subject 107, although in some embodiments, this step may be omitted. NK cells may then be harvested from a donor 109a or harvested from the subject 109b. After suitable treatment (such as, for example, expansion, treatment with cytokines, irradiation, and/or genetic modification), the harvested NK cells may be infused 111 into the subject. The process may end 115 here although, in some embodiments, a second suitable upregulating means (which may be the same as, or different from, the first upregulating means) is then employed to upregulate the cathelicidin gene CAMP in the subject 113. It will thus be appreciated that embodiments are possible which employ a first upregulating means only, a second upregulating means only, or both a first and second upregulating means. It will further be appreciated that upregulation of the cathelicidin gene CAMP expression in the subject may occur before, during and/or after NK cell therapy.

It will also be appreciated that, although cathelicidin induction is frequently disclosed herein as being an adjunct to NK cell therapy (and in particular, as a useful therapeutic step to be applied before, after, or simultaneously with NK cell therapy), more generally, cathelicidin induction may be an advantageous adjunct to various cancer therapies. These include, without limitation, CAR T cell therapy, radiation therapy, and chemotherapy.

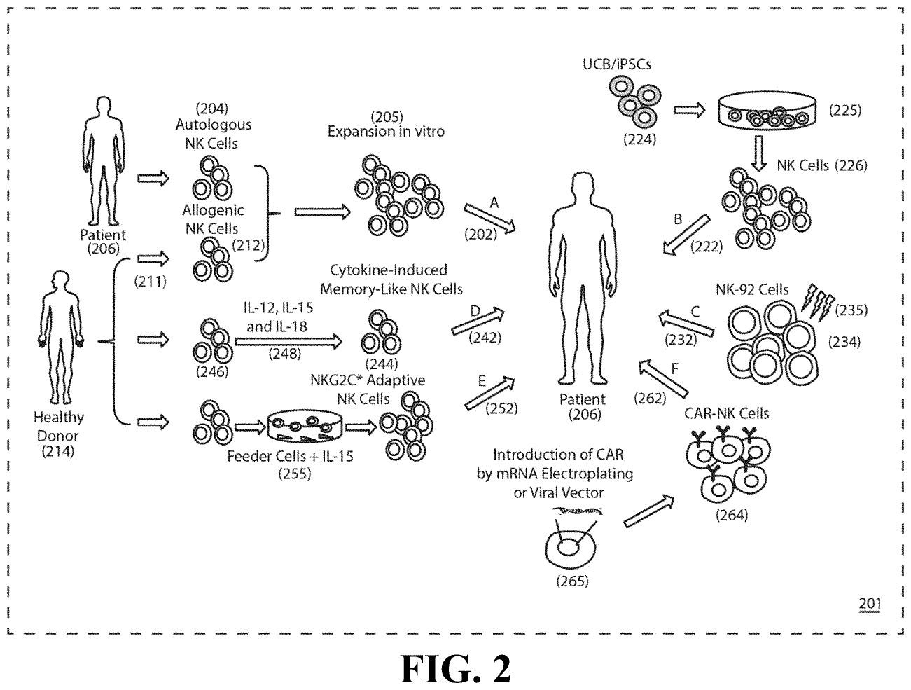

FIG. 2 illustrates some particular, non-limiting means 201 for sourcing or preparing NK cells that may be utilized in the systems and methodologies disclosed herein. In some embodiments, two or more of these methodologies may be utilized together. Similarly, in some embodiments, NK cells prepared by two or more of these methodologies may be administered to a patient, either separately or as a mixture.

In Scheme A 202, autologous NK cells 204 are harvested from a patient 206. The harvested cells are purified from peripheral blood (PB) and then expanded in vitro 205 through activation with suitable cytokines (such as, for example, IL-2 or IL-15). The expanded NK cells are then administered to the patient 206. In a variation of this method 211, allogeneic NK cells 212 with mismatched killer cell immunoglobulin-like receptors (KIRs) are harvested from a healthy donor 214, purified from PB and then activated with suitable cytokines 216 (such as, for example, IL-2 or IL-15) before administration into the patient 206. Of course, further variations of this embodiment are possible in which some NK cells 212 are harvested from the patient 206, and some are harvested from one or more donors 214. In these latter embodiments, the NK cells 212 may be administered to the patient separately or as a mixture.

In Scheme B 222, umbilical cord blood (UCB) and/or induced pluripotent stem cells (iPSCs) 224 are used as a source of functional NK cells 226. These NK cells may be co-cultured 225 with supportive feeder cells, or stimulated alone with a combination of cytokines, prior to administration to the patient. In some embodiments, this approach may utilize human iPSCs to produce NK cells with novel CARs that specifically target cancer cells in an antigen-specific manner.

In Scheme C 232, NK-92 cells 234 from the NK cell line are obtained. These cells are then irradiated 235 (for example, with 1000 cGy) prior to infusion. The use of NK-92 cells 234 in this approach is advantageous in that they display a robust and broad-spectrum cytotoxicity against malignant cells. Moreover, NK-92 cells are readily expanded under good manufacturing practice (GMP) conditions compared with allogeneic or UCB-derived NK cells. In addition, NK-92 cells may be efficiently manipulated with viral or non-viral vectors to enhance their targeting, homing, and killing activity. Finally, the safety of infusion with NK-92 cells has been confirmed in clinical trials.

In Scheme D 242, cytokine-induced memory-like NK cells 244 are obtained through pre-activation of human PB-derived NK cells 246 with one or more cytokines 248. Suitable cytokines may include, but are not limited to, IL-12, IL-15, and IL-18 and combinations thereof. The resulting cytokine-induced memory-like NK cells 244 are then administered to the patient.

In Scheme E 252, NKG2C⁺-adaptive NK cells 254 are preferentially expanded ex vivo from healthy donors. This may be achieved, for example, through culturing 255 with HLA-E-transfected 721.221 cells as feeder cells and a suitable cytokine such as, for example, IL-15. The resulting NKG2C⁺-adaptive NK cells 254 are then administered to the patient 206.

In Scheme F 262, NK cells are genetically modified with Chimeric Antigen Receptors (CARs) 264. This may occur, for example, through mRNA electroporation or viral vectors 265 in order to redirect the specificity and enhance the antitumor efficacy of the NK cell-based immunotherapy. The resulting CAR NK cells 264 are then administered to the patient 206.

As previously noted, in some embodiments of the systems and methodologies disclosed herein, upregulation of the cathelicidin gene CAMP in the subject may occur before, during and/or after NK T-cell therapy. In such embodiments, for example, a first method of upregulating the cathelicidin gene CAMP in the subject may be utilized before NK cell therapy, and a second method of upregulating the cathelicidin gene CAMP in the subject may be utilized after NK cell therapy. The first and second methods may be the same or different. By way of example but not limitation, in some embodiments, the second method may involve upregulating the cathelicidin gene CAMP in the subject at a lower level than the first method. In other embodiments, the second method may involve upregulating the cathelicidin gene CAMP in the subject in a different manner than that employed in the first method, or through the use of different materials.

As an example of the latter embodiment, a first pharmaceutical composition may be utilized to upregulate the cathelicidin gene CAMP in the subject prior to NK cell therapy, and a second pharmaceutical composition, which is distinct from the first pharmaceutical composition, may be utilized to upregulate the cathelicidin gene CAMP in the subject after NK cell therapy. The second pharmaceutical composition may differ from the first pharmaceutical composition in that, for example, the second pharmaceutical composition may upregulate the cathelicidin gene CAMP in the subject more weakly than the first pharmaceutical composition, or may be less cytotoxic than the first pharmaceutical composition. It will be appreciated that, in some embodiments, the upregulation of at least one protein product (which is preferably selected from the group consisting of hCAP-18 and LL-37 cathelicidin peptide) of CAMP gene in a subject may be verified before and/or after NK cell therapy, and this process may involve determining the levels or concentrations (relative or absolute) of these protein products in the blood of the patient or in the components thereof (here, it is noted that determining the concentrations or levels of these protein products in NK cells is especially preferred, since the concentrations or levels of these protein products in the blood serum of the patient may not be significantly or directly affected by the NK cell therapy).

Various means may be utilized in accordance with the teachings herein to upregulate the cathelicidin gene CAMP in a subject. These include, without limitation, the application to the subject of one or more pharmaceutical compositions of the type disclosed in U.S. Ser. No. 16/038,158, now published as U.S. 2019/0015361 (Barron et al.), “POLYTHERAPY MODULATING CATHELICIDIN GENE EXPRESSION MODULATION FOR THE TREATMENT OF ALZHEIMER'S DISEASE AND OTHER CONDITIONS”, which is incorporated herein by reference in its entirety, or the application to the subject of at least one substance selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, genistein and pharmaceutically acceptable salts thereof. One skilled in the art will appreciate that varying amounts of the foregoing compositions (or of various combinations of the foregoing compositions) may be required to achieve the desired level of upregulation of the cathelicidin gene CAMP in a particular subject or application, with the desired tolerability and minimal negative side effects.

Various other means may be utilized in accordance with the teachings herein to upregulate the cathelicidin gene CAMP in a subject. These include, without limitation, requiring the subject to undertake an exercise regime or to undergo radiation or light therapy, by vaccinating the subject with certain live vaccines (such as, for example, the bacille Calmette-Guerin (BCG) vaccine, or an oral poliovirus vaccine (OPV), which are known to provide upregulation of cathelicidin gene expression in human beings), or by causing the subject to undergo a therapy selected from the group consisting of (i) sauna therapy and (ii) hydrotherapy, wherein said therapy includes exposing the subject to a temperature of at least 74° C.

As previously noted, the NK cells utilized in various embodiments of the systems and methodologies disclosed herein may be obtained from various sources. For example, the NK cells may be obtained from the body of the subject to which they will be administered, or they may be obtained from one or more donors. Preferably, however, the NK cells are harvested from umbilical cord blood.

As previously noted, in some embodiments of the systems and methodologies disclosed herein, the NK cells may be genetically modified prior to being administered to a subject. More specifically, in some embodiments of the methodologies disclosed herein, CAMP gene expression may be upregulated in a donor. White blood cells (including NK cells) may be collected from that donor, and these white blood cells may be genetically transformed or modified through CAR (Chimeric Antigen Receptor) NK cell therapy (this therapy typically involves the addition of the gene for a special receptor that binds to a certain protein on the patient's cancer cells) to cause the NK cells to recognize or be receptive to particular markers or cellular ligands that may be present on cancer cells. Such markers may include, for example, PD-1 or PD-L1 (PD-1 is a protein found on T cells that regulates the body's immune responses in that, when it is bound to PD-L1 (another protein), it helps keep T cells from killing other cells, including cancer cells). The subject may then be treated using the methodologies disclosed herein for upregulating CAMP gene expression, after which the modified NK cells may be re-infused into the subject (here, it is noted that upregulated endogenous LL-37 expression will preferably have activated the subject's dendritic cells in such a way that NK cell therapy may work better). In other words, in this particular embodiment, if the donor is the subject, then the subject is treated to upregulated CAMP gene both before and after the steps of removing NK cells and then re-infusing transgenic NK cells.

The CAR NK cell therapy accomplishes two objectives. First of all, the NK cells are genetically altered so that they are receptive to the particular markers (e.g., PD-1 or PD-L1) found on cancer cells. Secondly, the NK cells are propagated in culture so there is a greater number of them. Hence, the success of the therapy is premised on the NK cells being focused on killing cancer cells in a subject, and on there being more of them.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

1. A method for treating a subject suffering from cancer, comprising:
diagnosing the subject as suffering from cancer;
upregulating the cathelicidin gene CAMP in the subject;
obtaining Natural Killer (NK) white blood cells; and
infusing the NK cells into the body of the subject.

2. The method of claim 1, wherein obtaining NK cells includes obtaining the NK cells from umbilical cord blood.

3. The method of claim 1, further comprising:
verifying the upregulation of the cathelicidin gene CAMP in the subject.

4. The method of claim 1, further comprising:
verifying the upregulation of at least one protein product of CAMP gene in the subject, wherein said at least one protein product is selected from the group consisting of hCAP-18 and LL-37 cathelicidin peptide.

5. The method of claim 4, wherein the upregulation of cathelicidin LL-37 is verified in the subject by (a) measuring the concentration of LL-37 in a first biological sample obtained from the subject prior to upregulating cathelicidin in the subject, (b) measuring the concentration of LL-37 in a second biological sample obtained from the subject after upregulating cathelicidin in the subject; and (c) comparing the concentration of cathelicidin in the first and second samples.

6. The method of claim 5, wherein upregulation of LL-37 is verified in the subject only if the concentration of cathelicidin in the second sample is at least 3 times the concentration of cathelicidin in the first sample.

7. The method of claim 5, wherein upregulation of LL-37 is verified in the subject only if the concentration of cathelicidin in the second sample is at least 4 times the concentration of cathelicidin in the first sample.

8. The method of claim 5, wherein upregulation of LL-37 is verified in the subject only if the concentration of cathelicidin in the second sample is at least 5 times the concentration of cathelicidin in the first sample.

9. The method of claim 1, further comprising:
verifying the upregulation by (a) obtaining a sample of blood from the subject, and (b) measuring the concentration of LL-37 in the blood sample.

10. The method of claim 1, further comprising:
verifying the upregulation by (a) obtaining peripheral monocytes from the subject, and (b) measuring the levels of CAMP gene mRNA, or the concentration of LL-37 in the obtained peripheral monocytes.

11. The method of claim 1, wherein diagnosing the subject as suffering from cancer includes diagnosing the subject as suffering from glioma.

12. The method of claim 1, wherein diagnosing the subject as suffering from cancer includes diagnosing the subject as suffering from pediatric diffuse glioma.

13. The method of claim 1, wherein diagnosing the subject as suffering from cancer includes diagnosing the subject as suffering from pediatric diffuse glioma characterized by a histone H3-K27M mutation.

14. The method of claim 1, further comprising:
modifying the NK cells such that they target a specific surface marker present on cancer cells of the cancer the patient has been diagnosed with, thereby obtaining modified NK cells;
wherein infusing NK cells into the body of the subject includes infusing the modified NK cells into the body of the subject.

15. The method of claim 13, wherein the surface marker is GD2.

16. The method of claim 13, wherein the subject's cancer cells are diagnosed as presenting a noninflammatory tumor microenvironment.

17. The method of claim 1, wherein upregulating cathelicidin in the subject includes treating the subject with a vaccine that upregulates cathelicidin.

18. The method of claim 1, wherein upregulating cathelicidin in the subject includes administering a pharmaceutical composition to the subject that upregulates cathelicidin.

19. The method of claim 18, wherein the pharmaceutical composition includes at least one substance selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, and pharmaceutically acceptable salts thereof.

20. The method of claim 18, wherein the pharmaceutical composition includes at least two substances selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, and pharmaceutically acceptable salts thereof.

21-132. (canceled)
